(19) United States
(12) Patent Application Publication
(10) Pub. No.: US 2010/0330200 A1
(43) Pub. Date: Dec. 30, 2010

(54) COMBINATION OF A RETINOID AND A PLATINUM ANTICANCER AGENT

(75) Inventors: Paolo Carminati, Milano (IT); Claudio Pisano, Aprilia (IT); Loredana Vesci, Roma (IT); Franco Zunino, Milano (IT)

Correspondence Address: LUCAS & MERCANTI, LLP, 475 PARK AVENUE SOUTH, 15TH FLOOR, NEW YORK, NY, 10016, US

(73) Assignee: SIGMA-TAU INDUSTRIE FARMACEUTICHE RIUNITE S.P.A., Rome (IT)

(21) Appl. No.: 12/517,926

(22) PCT Filed: Dec. 11, 2007
(86) PCT No.: PCT/EP2007/063725

§ 371 Date: Sep. 29, 2009

(30) Foreign Application Priority Data
Dec. 22, 2006 (EP) 06127060.9

(51) Int. Cl. A61K 33/24; A61P 35/00; A61P 35/04

(52) U.S. Cl. 424/649

(57) ABSTRACT

A combination comprising an atypical retinoid compound, preferably E-4-(3-(1-adamantyl)-4-hydroxyphenyl)cinnamic acid and an anticancer drug of the platinum family, preferably cisuplatin, together with a pharmaceutical composition comprising it and a kit for its administration in unitary or in coordinated sequential form. The kit preferably comprises a first formulation of E-4-(3-(1-adamantyl)-4-hydroxyphenyl)cinnamic acid dissolved in a mixture of ethanol and cremophor, and susupended in a saline solution, and a second formulation of cisuplatin in saline solution. The combination is used for the preparation of a medicament to inhibit tumor growth and tumor migration, in particular for the treatment of ovarian tumor and/or carcinoma.

The present invention relates to the pharmaceutical field and in particular to the combined use of a retinoid, in particular an atypical retinoid and an antitumor drug of the platinum family for the inhibition of tumor growth and migration, in particular for the treatment of ovarian tumor and/or carcinoma, breast cancer, pharynx carcinoma or in particular for the treatment of bone metastasis, more in particular from breast cancer.

### BACKGROUND OF THE INVENTION

It is well known in the art that synthetic retinoids, also known as retinoid-related molecules or atypical retinoids, can induce apoptosis by a mechanism that is independent of retinoid receptor signalling pathway [Lotan R., J. Biol. Regul. Homeost. Agents, 2003; 17, 12-28; Garattini E., Gianni M, Terao M., Curr. Pharm. Des., 2004; 10:433-448.].

Synthetic retinoids, in particular the ones containing the adamantyl moiety represent a series of potentially useful agents characterized by proapoptotic activity in a large variety of tumor cells and antitumor activity with minimal acceptable side effects [Dallavalle S, Zunino F., Expert Opin. Ther. Patents, 2005; 15:1625-1635; Cincinelli R., Dallavalle S., Merlini L. et al., J. Med. Chem., 2003; 46:909-912; Pisano C., Merlini L, Penco S. et al., J. Chemother., 2004; 16:74-76; Parrella E., Gianni M., Fratelli M. et al., Mol. Pharmacol., 2006; 70:909-924].

Recently, the present inventors reported that a novel adamantyl retinoid, (2E)-3-[3′-(1-adamantyl)-4′-hydroxy-[1,1′-biphenyl]-4-yl]-2-propenoic acid, hereinafter shortly ST1926, is a potent inducer of apoptosis in ovarian carcinoma cells [WO 03/011808; Parrella E., Gianni M., Fratelli M., et al., Mol. Pharmacol., 2006; 70:909-924].

The molecular mechanism involved in said apoptosis induction is not yet clearly defined, but the pattern of cellular response to ST1926 suggested the involvement of genotoxic stress as a relevant aspect of the mechanism of the proapoptotic activity [Parrella E., Gianni M., Fratelli M., et al., Mol. Pharmacol., 2006; 70:909-924].

It is known that a typical feature of cellular response to ST1926 is the activation of p53 and the modulation of genes involved in DNA damage response, however, both p53-dependent and—independent pathways appear to be implicated in apoptosis induction by ST1926 [Parrella E., Gianni M., Fratelli M. et al., Mol. Pharmacol., 2006; 70:909-924.].

Indeed, a number of molecular events could be involved in cellular response to adamantyl retinoids, but the critical determinants remain to be identified [Zuco V., Zanchi C., Lanzi C. et al., Neoplasia 2005; 7:667-677].

Antitumor activity of a number of adamantyl retinoids, including CD437, MX3350-1 and ST1926, has been described in human tumor models of various histotypes [Dallavalle S., Zunino F., Expert Opin., 2005; 15:1625-1635]. Thus, these retinoid-related molecules represent an emerging group of promising antitumor agents endowed with remarkable apoptotic activity against leukaemia cells and cells from various solid tumors [Dallavalle S., Zunino F., Expert Opin. Ther. Patents, 2005; 15:1625-1635; Pratesi G., Tortoreto M., Zunino F., Reg. Cancer Treat. 1990; 3:40-43].

ST1926 showed to induce high level of apoptosis in ovarian carcinoma cells at sub-micromolar concentrations [Parrella E., Gianni M., Fratelli M. et al., Mol. Pharmacol., 2006; 70:909-924].

Although the detailed mechanism of action of ST1926 and related molecules remains to be defined, several lines of evidence support that the genotoxic stress is a critical event mediating drug-induced apoptosis [Zuco V., Zanchi C., Cassinelli G. et al., Cell Death Diff., 2004; 11:280-289; Zuco V., Zanchi C., Lanzi C. et al., Neoplasia 2005; 7:667-677; Ortiz M. A., Bayon Y., Lopez-Hernandez F. J., Piedrafita F. J., Drug. Resistance Updates, 2002; 5:162-175].

Meco et al. (18^{th} Symposium on Molecular Targets and Cancer Therapeutics, 7-10 Nov., 2006) disclose a combination of ST 1926 and cisplatin and show a synergistic effect in the combined treatment of neuroblastoma tumors which do not respond to single therapies.

Lovat Penny et al. (International Journal of Cancer, 88 (6), 15 Dec. 2000, 977-985) report a study on different combinations of atypical retinoids and antitumor drugs. Experiments are described on the following combinations: cisplatin/fenretinide, etoposide/fenretinide, carboplatin/fenretinide, cisplatin/CD437, carboplatin/CD437 and etoposide/CD437. These combinations show a synergistic effect.

The present inventors investigated the efficacy of atypical retinoids in combination with anticancer drugs of the platinum family in comparison with the retinoid alone, since it is known that platinum anticancer agents are DNA damaging agents, and among the most effective agents used in standard first-line therapy of ovarian carcinoma.

The problem solved by the present invention is to enhance the efficacy of an atypical retinoid derivative and also provide a new chemotherapy combination able to overcome the side effects of the commonly used chemotherapy protocols, with a particular interest in reducing toxicity.

All malignant tumors are potentially capable of metastasizing to bone. However, bone metastases are found most commonly in breast and lung cancer and in prostate, thyroid, and renal cell carcinomas. Bone metastasis is generally a sign of incurable disease and also causes significant morbidity through pain and pathologic fractures. Bone metastases originate in the same way as other metastases: the tumor releases cells that are transported to distant organs by the circulation. The cells enter the bone marrow and are deposited there. Most cells will die but a few will proliferate and develop into clinically significant bone metastases (Mundy, 2001, Semin Oncol. 28: 2-8). Osteolytic bone metastases, in which bone resorption is mediated through the osteoclast, occur with several tumor types such as lung, renal and thyroid, but most frequently in breast cancer. However, in most cases of prostate cancer and sometimes in breast cancer, osteoblastic bone metastases develop.

A further problem solved by the present invention is to provide a treatment for bone metastasis, in particular from breast cancer.

### SUMMARY OF THE INVENTION

It has unexpectedly been found that the combined use of an atypical retinoid compound, such as the compounds of formula (I) as detailed below, and an anticancer drug of the platinum family resulted in an improved efficacy in terms of tumor growth without substantial increase of toxicity. In particular, the best results were achieved against ovarian carcinoma, such as the HOC18 ovarian carcinoma tumor and the IGROV-1 ovarian carcinoma line. Remarkable results were also obtained in other ovarian carcinoma lines, in particular platin-resistant forms, such as cisplatin-resistant forms, for example A2780/DDP; taxol-resistant forms, such as paclitaxel-resistant forms, for example 1A9PTX22.

Surprising results were also achieved in breast cancer, for example MDA-MB231, and also in squamous pharynx carcinoma, for example FaDu line. It is an object of the present invention the use of a combination of an atypical retinoid compound, in particular a compound of formula (I) as detailed below with an anticancer drug of the platinum family for the preparation of a medicament for the treatment of ovarian tumor and/or carcinoma, drug-resistant forms included, breast cancer and squamous pharynx carcinoma.

It is a related object of the present invention the use of a combination of an atypical retinoid compound, in particular a compound of formula (I) as detailed below with an anticancer drug of the platinum family for the preparation of a medicament for the treatment bone metastasis, in particular from breast cancer.

The present invention will be now described in detail also by means of figures and examples.

### DETAILED DESCRIPTION OF THE INVENTION

In the context of the present invention, the term “atypical retinoid” is well-known in the art of medicine, in particular of cancer therapy. Retinoid-related molecules (RRMs) are molecules endowed with remarkable and selective apoptotic activity against various leukaemia and cancer cell types (Garattini E., Terao M., J. Chemother., 2004, Nov. 16, Suppl. 4; 70-3; Garattini E., Gianni N., Terao M., Curr. Pharm. Des., 2004, 10 (4); 433-438; Ralhan R., Kaur J., J. Biol. Regul. Homeost. Agents, 2003, January-March; 17(1), 66-91; Simoni D., Tolomeo M., Curr. Pharm. Des., 2001, Nov. 7 (17); 1823-37).

The retinoid is a compound of formula (I)

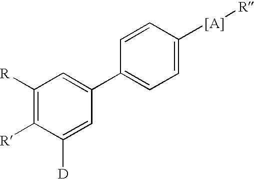

wherein:

R represents alkyl, cycloalkyl, heterocycloalkyl, phenyl, phenyl substituted, adamantyl wherein at least one of the CH can be substituted with C-halogen or C-alkyl and one of the CH₂ can be substituted by O, S, CH-halogen, CH-aryl, CH-heteroaryl, CH-arylalkyl, CH-heteroarylalkyl, CH-amino;

R′ represents OR′″, OCOR′″, COR^{IV};

R′-D represents O—(CH₂)ₙ—O; where n=1-3;

D represents H, OH, O-alkyl, (CH₂)ₙ—NH₂, (CH₂)ₙ—-NH-alkyl, (CH₂)ₙ—OH, where n=1-4;

R″ represents tetrazole, SO₃H, NHSO₃H, CHO, COOH, CONHOH, CONH-aryl, CONH—C₆H₄OH, CH₂OR′″; PO₃H₂; CO—(CH₂)ₙ-aryl, where n=0-4;

R′″ represents H, alkyl, aryl, arylalkyl, heteroaryl, heteroarylalkyl, SO₃H, α or β D- and L-glycosyl;

R^{IV} represents H, OH, OR′″;

[A] represents [C(R^{V},R^{VI})—C(R^{VII},R^{VIII})]ₙ, [C(R^{IX})═C(R^{X})]ₙ, [C≡C]ₙ, where n=0-3;

R^{V}, R^{VI}, R^{VII}, R^{VIII} represent H, alkyl, halogen, OH, OR′″, NO₂, NH₂, aryl, —O—, —CH₂—, CX₂— (where X is halogen), —CH(R′″)—;

R^{IX}, R^{X} represent H, OH, halogen, alkyl, aryl, CN, NO₂, COOR′″.

The above compounds are disclosed in WO 03/011808, both for their preparation and pharmacological profile. Specific reference is made to this document for disclosure of the compounds of formula (I).

Preferred compounds of formula (I) are comprised in the following group 4-(3-(1-Adamantyl)-4-tert-butyldimethyl-silyloxyphenyl)benzaldehyde;

methyl E-4(3-(1-Adamantyl)-4-tert-butyldimethyl-silyloxyphenyl)cinnamate;

methyl E-4-(3-(1-adamantyl)-4hydroxyphenyl)cinnamate;

E-4-(3-(1-adamantyl)-4-hydroxyphenyl)cinnamic acid (ST 1926);

methyl 4-(3-(1-adamantyl)-4-methoxyphenyl)propionate;

4-(3-(1-adamantyl)-4-methoxyphenyl)propionic acid (ST 1879);

E-4-(3-(1-adamantyl)-4-methoxyphenyl)cinnamyl alcohol;

methyl E-4-(4-hydroxyphenyl)cinnamate;

methyl E-4-(3-(1-methylcyclohexyl)-4-hydroxyphenyl)cinnamate;

2-(1-adamantyl)-4-bromo-6-N-phthalimidomethyl)phenol;

methyl E-4-(3-(1-adamantyl)-5-(N-phthalimidomethyl)-4-hydroxyphenyl)cinnamate;

E-4-(3-(1-adamantyl)-5-(N-phthalimidomethyl)-4-hydroxyphenyl)cinnamic acid;

E-4-(3-(1-adamantyl)-5-(aminomethyl)-4-hydroxyphenyl)cinnamic acid;

4-(7-adamantan-1-yl-benzo(1,3)dioxol-5-yl)-benzaldehyde;

methyl E-4-(7-adamantan-1-yl-benzo(1,3)dioxol-5-yl)-cinnamate;

E-4-(7-adamantan-1-yl-benzo(1,3)dioxol-5-yl)-cinnamic acid;

methyl 2-[4-(3-(1-adamantyl)-4-hydroxyphenyl)]cyclopropanecarboxylate;

cis and trans 2-(4-(3-(1-adamantyl)-4-hydroxyphenyl)]cyclopropanecarboxylic acids;

methyl E-4-(3-(1-adamantyl)-4-methoxyphenyl)cinnamate;

E-4-(3-(1-adamantyl)-4-methoxyphenyl)cinnamic acid (ST 1898);

A particularly preferred compound of formula (I) is E-4-(3-(1-adamantyl)-4-hydroxyphenyl)cinnamic acid (hereinafter briefly named ST 1926).

Anticancer agents of the platinum family are well known in the art and do not need any special indication. Anticancer agents currently used in clinical practice are disclosed in national pharmacopoeias, for example European Pharmacopoeia, United States Pharmacopoeia, or textbooks and reviews, see for example Holland Frei Cancer Medicine 6 (September 2003, Section 12, Chpt. 51); Abu-Surrah A S, Kettunen M., Curr. Med. Chem. 2006; 13(11): 1337-57; McKeage M. J., Expert Opin. Investig. Drugs, 2005, Aug. 14(8): 1033-46, and the references cited in all these publications. Moreover, patent literature is abundant of disclosure on platinum compounds in the treatment of cancer.

Preferred anticancer agents of the platinum family are cisplatin, carboplatin and oxaliplatin.

The most preferred combination is E-4-(3-(1-adamantyl)-4-hydroxyphenyl)-cinnamic acid (ST 1926) and cisplatin.

The combination elements (retinoid compound—component (a)—and platinum anticancer agent—component (b)) can be administered together, one after the other or separately in one combined unit dosage form or in two separate unit dosage forms. The unit dosage form may also be a fixed combination.

It will be understood that references to the components (a) and (b) are meant to also include the pharmaceutically acceptable salts of any of the active substances. If active substances comprised by components (a) and/or (b) have, e.g., at least one basic center, they can form acid addition salts.

Corresponding acid addition salts can also be formed having, if desired, an additionally present basic center. Active substances having an acid group, e.g., COOH, can form salts with bases. The active substances comprised in components (a) and/or (b) or a pharmaceutically acceptable salts thereof may also be used in form of a hydrate or include other solvents used for crystallization, also known as solvates.

Pharmaceutically acceptable salts and solvates are well-known to the skilled person and no further explanation is necessary. See for example Wermuth, C. G. e Stahl, P. H. (eds.) Handbook of Pharmaceutical Salts, Properties; Selection and Use; Verlag Helvetica Chimica Acta, Zürich, 2002.

Any of the combination of components (a) and (b), the method of treating a warm-blooded animal comprising administering these two components, a pharmaceutical composition comprising these two components for simultaneous, separate or sequential use, the use of the combination for the delay of progression or the treatment of a proliferative disease or for the manufacture of a pharmaceutical preparation for these purposes or a commercial product comprising such a combination of components (a) and (b), all as mentioned or defined above, will be referred to subsequently also as combination of the invention (so that this term refers to each of these embodiments which thus can replace this term where appropriate).

Simultaneous administration may, e.g., take place in the form of one fixed combination with two or more active ingredients, or by simultaneously administering two or more active ingredients that are formulated independently. Sequential use (administration) preferably means administration of one (or more) components of a combination at one time point, other components at a different time point, that is, in a chronically staggered manner, preferably such that the combination shows more efficiency than the single compounds administered independently (especially showing synergism). Separate use (administration) preferably means administration of the components of the combination independently of each other at different time points.

Also combinations of two or more of sequential, separate and simultaneous administration are possible, preferably such that the combination component-drugs show a joint therapeutic effect that exceeds the effect found when the combination component-drugs are used independently at time intervals so large that no mutual effect on their therapeutic efficiency can be found, a synergistic effect being especially preferred.

The term “delay of progression”, as used herein, means administration of the combination to patients being in a pre-stage or in an early phase, of the first or subsequent manifestations; or a relapse of the disease to be treated in which patients, e.g., a pre-form of the corresponding disease is diagnosed; or which patients are in a condition, e.g., during a medical treatment or a condition resulting from an accident, under which it is likely that a corresponding disease will develop.

“Jointly therapeutically active” or “joint therapeutic effect” means that the compounds may be given separately (in a chronically staggered manner, especially a sequence-specific manner) in such time intervals that they preferably, in the warm-blooded animal, especially human, to be treated, still show a (preferably synergistic) interaction (joint therapeutic effect).

“Pharmaceutically effective” preferably relates to an amount that is therapeutically or in a broader sense also prophylactic ally effective against the progression of a proliferative disease. Such amount is found through normal clinical trials, whose design is within the skills of the person of ordinary experience in this field. See also EMEA or FDA Guidelines.

The term “a commercial package” or “a product”, as used herein defines especially a “kit of parts” in the sense that the components (a), which is the retinoid derivative and (b), which includes the anticancer agent of the platinum family, (b) this implies only 2 component-combinations we need to also state that one or more compounds can be used in the “combination” as defined above, can be dosed independently or by use of different fixed combinations with distinguished amounts of the components (a) and (b), i.e., simultaneously or at different time points. Moreover, these terms comprise a commercial package comprising (especially combining) as active ingredients components (a) and (b), together with instructions for simultaneous, sequential (chronically staggered, in time-specific sequence, preferentially) or (less preferably) separate use thereof in the delay of progression or treatment of a proliferative disease. The parts of the kit of parts can then, e.g., be administered simultaneously or chronologically staggered, that is at different time points and with equal or different time intervals for any part of the kit of parts. Very preferably, the time intervals are chosen such that the effect on the treated disease in the combined use of the parts is larger than the effect which would be obtained by use of only any one of the combination partners (a) and (b) as can be determined according to standard methods. The ratio of the total amounts of the combination partner (a) to the combination partner (b) to be administered in the combined preparation can be varied, e.g., in order to cope with the needs of a patient sub-population to be treated or the needs of the single patient which different needs can be due to the particular disease, age, sex, body weight, etc. of the patients. Preferably, there is at least one beneficial effect, e.g., a mutual enhancing of the effect of the combination partners (a) and (b), in particular, a more than additive effect, which hence could be achieved with lower doses of each of the combined drugs, respectively, than tolerable in the case of treatment with the individual drugs only without combination, producing additional advantageous effects, e.g., less side effects or a combined therapeutic effect in a non-effective dosage of one or both of the combination partners (components) (a) and (b), and very preferably a strong synergism of the combination partners (a) and (b).

Both in the case of the use of the combination of components (a) and (b) and of the commercial package, any combination of simultaneous, sequential and separate use is also possible, meaning that the components (a) and (b) may be administered at one time point simultaneously, followed by administration of only one component with lower host toxicity either chronically, e.g., more than 3-4 weeks of daily dosing, at a later time point and subsequently the other component or the combination of both components at a still later time point (in subsequent drug combination treatment courses for an optimal anti-tumor effect) or the like.

The combination of the invention can also be applied in combination with other treatments, e.g., surgical intervention, hyperthermia and/or irradiation therapy.

The combination of the invention will generally be administered in a suitable formulation. Such formulation takes the form of conventional pharmaceutical compositions.

The pharmaceutical compositions according to the present invention can be prepared by conventional means and are those suitable for enteral, such as oral or rectal, and parenteral administration to mammals including man, comprising a therapeutically effective amount of a retinoid derivative and at least one chemotherapeutic agent of the platinum family alone or in combination with one or more pharmaceutically acceptable carriers, especially those suitable for enteral or parenteral application for component (a) and injectable application for component (b).

The pharmaceutical compositions comprise from about 0.00002% to about 100%, especially, e.g., in the case of infusion dilutions that are ready for use) of 0.0001-0.02%, or, e.g., in case of injection or infusion concentrates or especially parenteral formulations, from about 0.1% to about 95%, preferably from about 1% to about 90%, more preferably from about 20% to about 60%, active ingredient (weight by weight, in each case).

Pharmaceutical compositions according to the invention may be, e.g., in unit dose form, such as in the form of ampoules, vials, dragées, tablets, infusion bags or capsules.

The effective dosage of each of the combination partners employed in a formulation of the present invention may vary depending on the particular compound or pharmaceutical compositions employed, the mode of administration, the condition being treated and the severity of the condition being treated. A physician, clinician or veterinarian of ordinary skill can readily determine the effective amount of each of the active ingredients necessary to prevent, treat or inhibit the progress of the condition.

The dose for component (A) may range from 100-1500 mg daily, e.g., 200-1000 mg/day, such as 200, 400, 500, 600, 800, 900 or 1000 mg/day, administered in one or two doses daily.

The dose for component (b) may be administered to a human according to standard protocols.

Pharmaceutical preparations for the combination therapy for enteral or parenteral administration are, e.g., those in unit dosage forms, such as sugar-coated tablets, capsules or suppositories; and furthermore ampoules. If not indicated otherwise, these formulations are prepared by conventional means, e.g., by means of conventional mixing, granulating, sugar-coating, dissolving or lyophilizing processes. It will be appreciated that the unit content of a combination partner contained in an individual dose of each dosage form need not in itself constitute an effective amount since the necessary effective amount can be reached by administration of a plurality of dosage units. One of skill in the art has the ability to determine appropriate pharmaceutically effective amounts of the combination components.

Preferably, the compounds or the pharmaceutically acceptable salts thereof, are administered as an oral pharmaceutical formulation in the form of a tablet, capsule or syrup; or as parenteral injections if appropriate.

In preparing compositions for oral administration, any pharmaceutically acceptable media may be employed, such as water, glycols, oils, alcohols, flavouring agents, preservatives or colouring agents. Pharmaceutically acceptable carriers include starches, sugars, microcrystalline celluloses, diluents, granulating agents, lubricants, binders and disintegrating agents.

Solutions of the active ingredient, and also suspensions, and especially isotonic aqueous solutions or suspensions, are useful for parenteral administration of the active ingredient, it being possible, e.g., in the case of lyophilized compositions that comprise the active ingredient alone or together with a pharmaceutically acceptable carrier, e.g., mannitol, for such solutions or suspensions to be produced prior to use. The pharmaceutical compositions may be sterilized and/or may comprise excipients, e.g., preservatives, stabilizers, wetting and/or emulsifying agents, solubilizers, salts for regulating the osmotic pressure and/or buffers, and are prepared in a manner known per se, e.g., by means of conventional dissolving or lyophilizing processes. The solutions or suspensions may comprise viscosity-increasing substances, such as sodium carboxymethylcellulose, carboxymethylcellulose, dextran, polyvinylpyrrolidone or gelatin. Suspensions in oil comprise as the oil component the vegetable, synthetic or semi-synthetic oils customary for injection purposes.

The isotonic agent may be selected from any of those known in the art, e.g., mannitol, dextrose, glucose and sodium chloride. The infusion formulation may be diluted with the aqueous medium. The amount of aqueous medium employed as a diluent is chosen according to the desired concentration of active ingredient in the infusion solution. Infusion solutions may contain other excipients commonly employed in formulations to be administered intravenously, such as antioxidants.

Any other information useful to prepare a pharmaceutical composition for carrying out the present invention can be drawn out from the general common knowledge of the skilled person, from example general textbooks, such as Remington's Pharmaceutical Sciences, Mack. Pub.

The combination of the present invention can also be in the form of “a combined preparation”, which, as used herein, defines especially a “kit of parts” in the sense that the combination partners (a) and (b) as defined above can be dosed independently or by use of different fixed combinations with distinguished amounts of the combination partners (a) and (b), i.e., simultaneously or at different time points. The parts of the kit of parts can then, e.g., be administered simultaneously or chronologically staggered, that is at different time points and with equal or different time intervals for any part of the kit of parts. The ratio of the total amounts of the combination partner (a) to the combination partner (b) to be administered in the combined preparation can be varied, e.g., in order to cope with the needs of a patient sub-population to be treated or the needs of the single patient based on the severity of any side effects that the patient experiences.

In a preferred embodiment of the present invention, the kit of parts for the coordinated administration includes a first formulation of ST1926 dissolved in a mixture of ethanol and cremophor, and suspended in a saline solution (ethanol-cremophor), and a second formulation of cisplatin in saline solution to be sequentially administered to the subject in need of this administration.

In a more preferred embodiment, in said kit, said first formulation comprises ST1926 dissolved in a mixture of 50% ethanol and 50% cremophor (50+50%) and suspended in a saline solution with 10% final concentration of ethanol-cremophor, and said second formulation comprising cisplatin diluted in saline solution, both said first formulation and said second formulation to be sequentially administered to said subject, each in a volume of 10 ml/kg of body weight.

The kit provided by the present invention and the related formulations can be prepared according to well-known techniques within the general knowledge of the person of ordinary skill in the art. Cremophor® (polyoxyethylenglyceroltri-ricinoleat 35) is a commercially available product, for example is marketed by BASF and is well-known in the formulation of antitumor drugs, see for example US2005191323 and cited references. Cisplatin is a well-known anticancer drug, and its formulations are within the skill of the person with ordinary experience in this field, see for example United States Pharmacopoeia.

Administration will be carried out according to the decision of the medical doctor, according to type of the tumor to be treated, patient's conditions and any other considerations pertaining the exercise of medical science.

The present invention also relates to the use of the combination herein disclosed for the preparation of a medicament. In particular, the medicament is intended for treating tumor and/or tumor metastasis. In a further particular embodiment, the medicament provided by the present invention is suitable for the treatment of drug-resistant tumor and/or carcinoma. In a preferred embodiment, the tumor is ovarian tumor and/or carcinoma, whether drug-resistant or not. Exemplary tumors treated according to the present invention are HOC18, IGROV-1, A2780/DDP, 1A9PTX22.

It is understood that other ovarian tumors responding to the combination herein disclosed fall within the terms and teaching of the present invention. In another preferred embodiment, the present invention also relates to the use of the combination herein disclosed for the preparation of a medicament intended for treating breast cancer. Exemplary breast cancer treated according to the present invention is MDA-MB231.

It is understood that other breast cancers responding to the combination herein disclosed fall within the terms and teaching of the present invention.

In still another preferred embodiment, the present invention also relates to the use of the combination herein disclosed for the preparation of a medicament intended for treating pharynx carcinoma. Exemplary pharynx carcinoma treated according to the present invention is FaDu.

It is understood that other pharynx carcinomas responding to the combination herein disclosed fall within the terms and teaching of the present invention.

Treatment of tumors falls within the practice of the skilled person and no particular instructions are needed in this context. Exemplary treatment is provided in the example, describing one way of carrying out the invention on an animal model which is universally accepted in the field of cancer drug development. Reference can be made to common manuals and textbooks, such as, for example The Merck Manual of Diagnosis and Therapy and the references cited therein. Drug resistance is intended in the context of the present invention as the resistance or diminished response of a neoplasm to an antineoplastic agent in a subject suffering from a cancer, see for example Holland Frei Cancer Medicine 6 (September 2003, Section 11, Chpt. 48)

In a preferred embodiment of the present invention, the antitumor agent of the platinum family is administered according to standard protocols and the retinoid is administered after the platinum agent, for example 1 hour after the administration of the antitumor platinum agent.

According to the present invention, both pre-treated and untreated patients can be elected for the treatment with the combination herein disclosed. For example, patients suffering from ovarian tumor underwent epirubicin/cyclophosphamide treatment or any other treatment conventionally used in this type of therapy. Patients suffering from tumor resistant to conventional therapy, in particular platinum therapy are also eligible for the treatment according to the present invention.

The atypical retinoid according to the present invention, in particular a compound of the formula (I) as above disclosed, is administered through a conventional pharmaceutical formulation. In an embodiment of such formulation, the compound of formula (I), for example ST1926, is dissolved in a mixture of ethanol and cremophor (50+50%) and suspended in saline (ethanol-cremophor 10% final concentration). The antitumor drug of the platinum family, for example cisplatin, is also administered through a conventional pharmaceutical formulation, for example is diluted in saline.

The therapeutical protocol is established by the medical doctor, according to the experience and knowledge of the ordinary practitioner.

Mutatis mutandis, the same considerations apply to the other embodiments of the present invention, in particular to the treatment of breast cancer and pharynx carcinoma, as well as to bone metastasis.

Further features of the present invention will be evident with reference to the following examples and attached figures wherein:

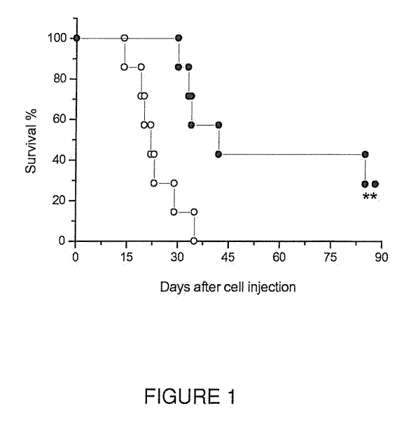

FIG. 1 shows the survival curves of mice after daily i.p. injection, for 5 days/week for 4 weeks, of IGROV-1 ovarian carcinoma cells (Kaplan-Meier plot); vehicle-treated mice are represented with (◯); mice treated with ST1926 10 mg/kg are represented with (), experimental groups consisted of 7 mice. **P<0.01 by two-sided log-rank λ² test vs. vehicle-treated mice.

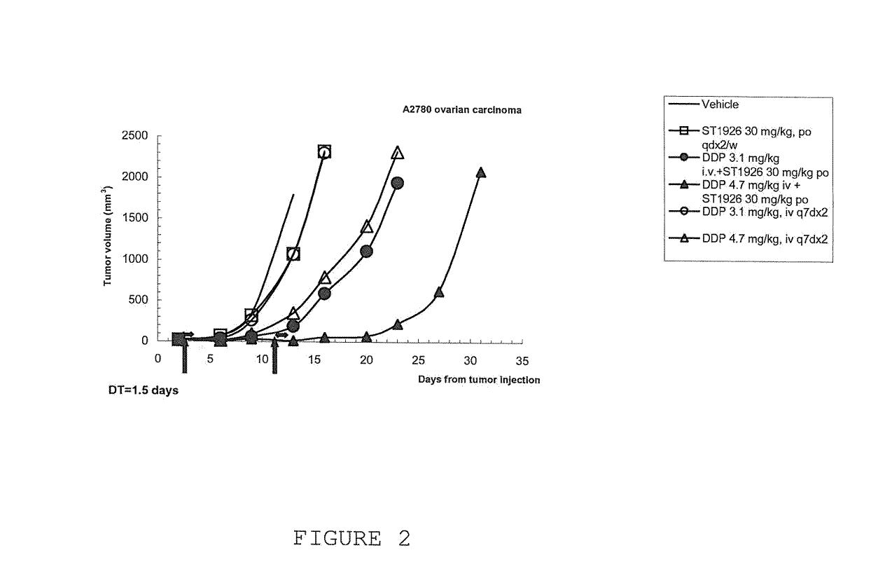

FIG. 2 shows the effect of ST1926, cisplatin and combination ST1926/cisplatin in A2780 ovarian carcinoma.

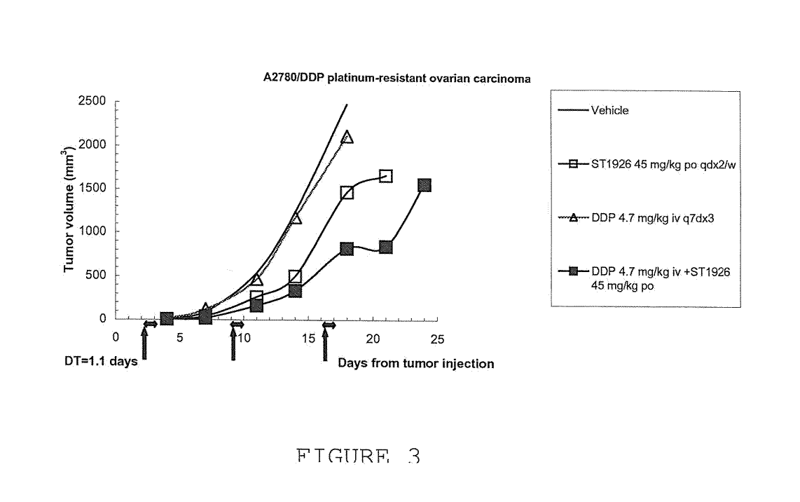

FIG. 3 shows the effect of ST1926, cisplatin and combination ST1926/cisplatin in A2780/DDP.

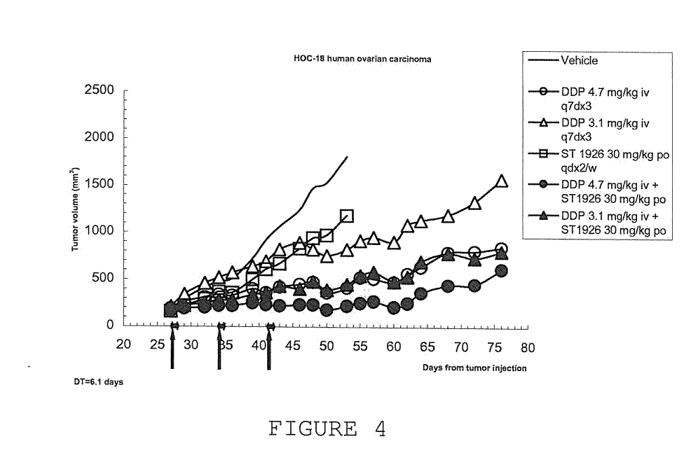

FIG. 4 shows the effect of ST1926, cisplatin and combination ST1926/cisplatin in HOC-18 ovarian carcinoma.

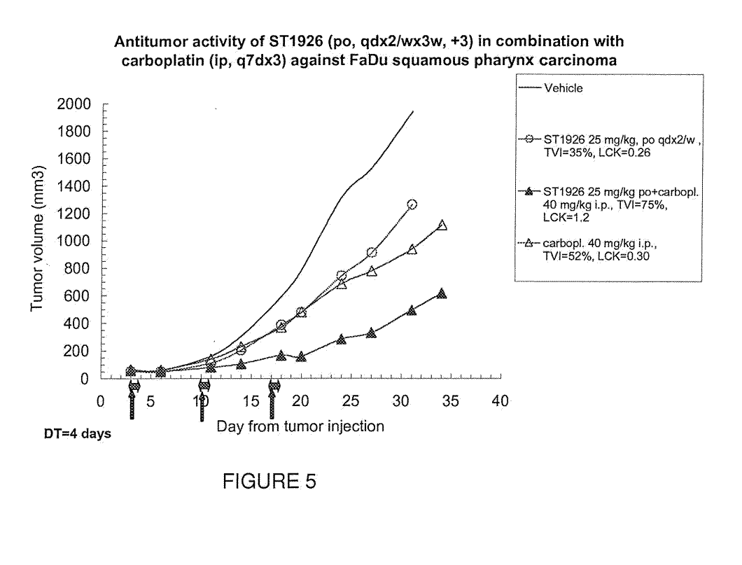

FIG. 5 shows the effect of ST1926, cisplatin and combination ST1926/cisplatin in the ovarian carcinoma paclitaxel-resistant 1A9PTX22.

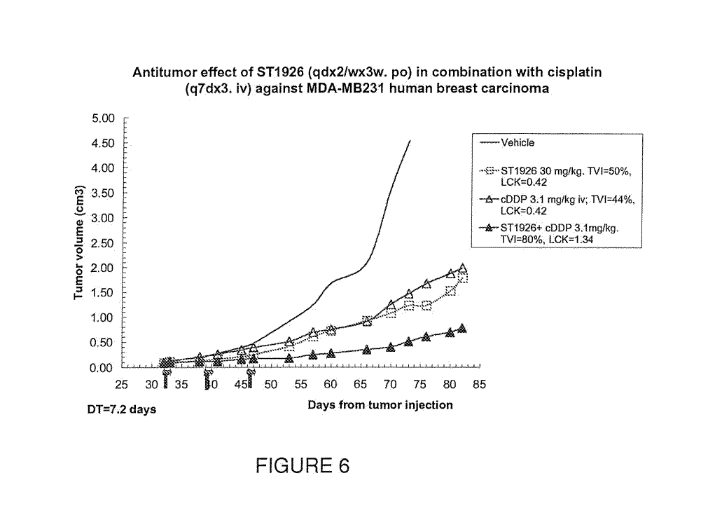

FIG. 6 shows the effect of ST1926, cisplatin and combination ST1926/cisplatin in the breast carcinoma MDA-MB 231.

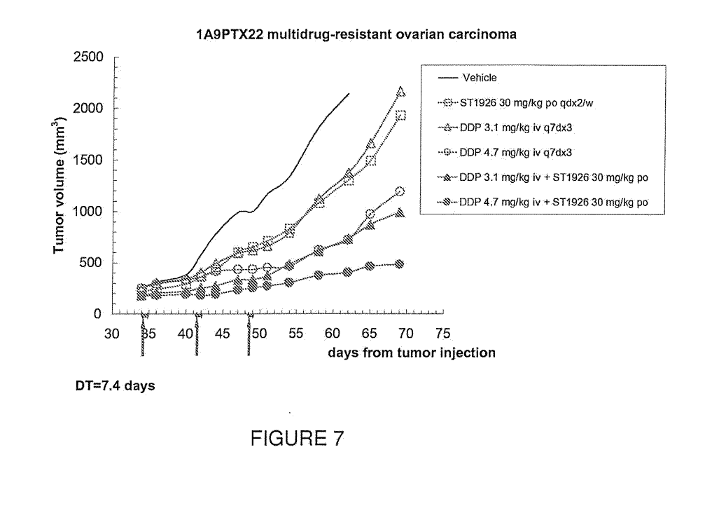

FIG. 7 shows the effect of ST1926, carboplatin and combination ST1926/carboplatin in the squamous pharynx carcinoma FaDu.

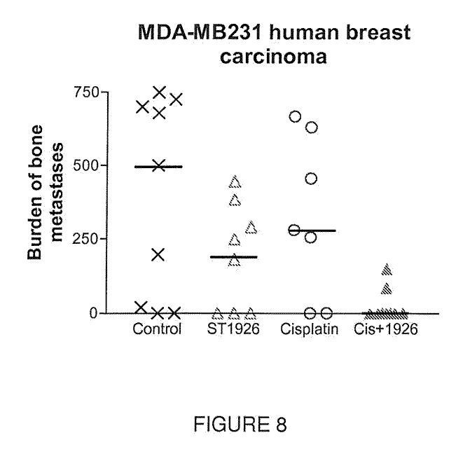

FIG. 8 shows the effect of ST1926, cisplatin and combination ST1926/cisplatin in bone metastases from breast carcinoma MDA-MB231.

The following examples further illustrates the invention.

### EXAMPLE 1

Ovarian Cancer

Four human ovarian tumor models were tested: HOC18 tumor line from a epirubicin/cyclophosphamide-pretreated patient [Nicoletti M. I., Colombo T., Rossi C. et al., Cancer Res., 2000; 60:842-846]; IGROV-1 cell lines derived from untreated patients [Eva A., Robbins K. C., Andersen P. R. et al., Nature, 1982; 295:116]; A2780/DDP cell line selected for resistance to cisplatin from untreated patients [Behrens B. C., Hamilton T. C., Masuda H., et al., Cancer Res., 1987; 47:414-418]; 1A9PTX22 cell line being a paclitaxel-resistant subline of the A2780 subclone 1A9 [Behrens B. C., Hamilton T. C., Masuda H. et al., Cancer Res., 1987; 47:414-418.].

The used animal model was female athymic Swiss nude mice, 10 weeks-old (Charles River, Calco, Italy), maintained in laminar flow rooms, kept at constant temperature and humidity and with free access to food and water.

The composition was prepared by dissolving ST1926 in a mixture of ethanol and cremophor (50+50%) and suspended in saline (ethanol-cremophor 10% final concentration) employing a magnetic stirrer, cisplatin (Platinex, Bristol Meyers Squibb) was diluted in saline and both were administered in a volume of 10 ml/kg of body weight.

For chemotherapy experiments, tumor fragments from tumor lines were obtained by subcutaneously inoculating exponentially growing tumor cells in nude mice and maintaining the human tumor lines by subcutaneous passages of tumor fragments (about 2×2×6 mm) in healthy mice, each group included four/five mice bearing bilateral tumors. Tumors were inoculated on day 0 and tumor growth was followed by biweekly measurements of tumor diameters with a Vernier caliper. Tumor volume (TV) was calculated according to the Formula: TV (mm³)=d²×D/2 where d and D are the shortest and the longest diameter, respectively. Drug treatment started at different tumor sizes. ST1926 was administered orally by gavage in a range of doses.

Cisplatin was delivered intravenously every seventh day for three times (q7d×3). In combination studies ST1926 was given one hour after cisplatin injection.

Drug Efficacy was Assessed as:

i) Tumor volume inhibition percentage (TVI %) in drug-treated versus control mice expressed as: TVI %=100-(mean TV treated/mean TV control×100). In the studies with drug combination the “expected TVI % value”, representing the TVI % due to an additive effect, was calculated by summing TVI % induced by cisplatin+TVI % induced by ST1926 on the surviving fraction of tumor.

ii) Log₁₀ cell kill (LCK) calculated by the formula: LCK=(T−C)/3.32×DT where T and C are the mean time (days) required for treated (T) and control (C) tumors, respectively, to reach a determined volume and DT is the doubling time of control tumors.

Toxic Effects of the Drug Treatment were Assessed as:

i) Body weight loss percentage (BWL %), calculated as: BWL %=100−(mean BW day x/mean BW day 1×100), where day 1 is the first day of treatment and day x is any day thereafter. The highest (max) BWL % is reported in the Tables.

ii) Lethal toxicity, assessed as deaths occurring in treated mice before the death of the first control mouse.

For statistical comparison of tumor volumes in treated vs. control mice the Student's t test (two-sided) was used.

The IGROV-1 ovarian carcinoma line was maintained by serial intraperitoneal passages in healthy mice. Tumor grows as ascites and small solid masses. IGROV-1 ascitic cells were collected and prepared as previously described [Pratesi G., Tortoreto M., Zunino F., Reg. Cancer Treat., 1990; 3:40-43] and 2.5×10⁶ cells/mouse (in 0.2 ml of saline) were injected intraperitoneally. Mice developed hemorrhagic and diffuse carcinomatosis and eventually died by 20 to 35 days. Animals were inspected daily and weighed three times a week. The median day of death (median survival time: MST) was considered as the experimental end point.

Experimental groups consisted of seven mice.

The compound was delivered intraperitoneally at a dose of 10 mg/kg, daily for 5 days a week for 4 weeks (qd×5/w×4w), starting the day after cell injection. Drug activity was assessed as increase in life span (ILS) %, i.e. the percentage increase of MST in treated (T) over control (C) mice (T/C×100−100). Mice alive at the end of experiment (day 88) without disease were considered “long term survivors” (LTS).

For statistical comparison, curves reporting the percentage of surviving animals over time were estimated by the Kaplan-Meier product limit method and compared with the log-rank test. The test was two-sided.

For assessing the tumor migration and metastasis formation process, Matrigel (BD Biosciences) was thawed on ice overnight at 4° C. and 200 μL (at 10 mg/ml) was spread per well of a 48-well plate. The plates were polymerized for 30 minutes at 37° C. Cells were counted using trypan blue exclusion to ensure viability and plated in culture medium without serum at a density of 3×10⁴ viable cells per well. At 5, 24 and 48 hours post seeding on Matrigel, tubulogenesis was observed by inverted microscope. In each condition, five randomly selected fields of view were photographed in each well. Images were captured using a Diagnostic Instruments digital camera attached to a microscope at ×4 magnification.

Tables 1 and 2 show the antitumor activity of ST1926 in a panel of human ovarian carcinoma xenografts growing sub cutaneous in female athymic mice. The retinoid was delivered per os according to various schedules. The efficacy of ST1926, 30 or 45 mg/kg, delivered 2 days/week (qd×2/w) for 2 or 3 weeks, was generally low in all the tumors investigated, never overcoming 50% of TV inhibition. Both doses were well tolerated. Against the IGROV-1 tumor, additional doses and schedules were investigated without substantial improvement in the efficacy, which remained moderate.

Using the 5 days/week (qd×5/w) schedule, the maximum tolerated dose was 15 mg/kg, whereas the dose of 20 mg/kg showed lethal toxicity.

In contrast to what observed against solid tumors, ST1926, 10 mg/kg, qd×5/w×4 weeks, showed a relevant antitumor activity in the locoregional treatment of intra peritoneal growing IGROV-1 tumor where it resulted in significantly (P<0.01) increased survival time over control mice and produced 2 out of 7 survivors (without evidence of tumor) at the end of experiment (day 88).

Cisplatin was delivered according to a weekly schedule (q7d), which is considered as the optimal one in our experimental models, and ST1926 was administered per os, the same day (1 h later) and the day after each cisplatin injection (qd×2/w) (FIG. 1).

Table 1 shows the results obtained in the tested tumors, characterized by variable sensitivity to cisplatin. A comparison of TVI % indicated that the combined use of cisplatin with ST1926 was able to achieve an antitumor effect more than additive in all of the 4 tumor lines investigated, even though at various levels.

Moreover, as shown in FIG. 2, the most sensitive tumor line to the combination was the cisplatin-sensitive A2780, where both TVI and LCK were strongly increased. Noteworthy the effect of the combination resulted more than additive even on the cisplatin-resistant A2780/DDP tumor, where a high LCK value was achieved, as shown in FIG. 3.

Against the HOC18 tumor, the combination was markedly effective as it is shown in FIG. 4, since it produced a substantial increase (5/23 tumors) of complete responses (i.e., no evidence of disease at the experiment end) over cisplatin-treated tumors (1/10 tumors). The increase of the efficacy of the combination was less marked in the treatment of the 1A9PTX22 tumor, in terms of TVI %, as evident from the results shown in FIG. 5. However, the combination of the higher dose of cisplatin with ST1926 resulted in a substantial increase in the LCK value. Concerning the toxicity of the combination, an increase in BWL was generally observed during treatment, but all mice recovered and no one died for toxicity.

TABLE 1

| | Treatment (mg/kg)^{b} | | | TVI %^{c} | Obs. | LCK^{d} | | |
|---|---|---|---|---|---|---|---|---|
| Tumor (D.T.)^{a} | 1^{st} day | Cisplatin | ST1926 | Exp. | (day) | (mm³) | BWL %^{e} | NED^{f} |
| A2780 | 2 | | 30 | | 40 | 0.4 | 5 | |
| (1.5) | | 3.1 | | | 40 | 0.4 | 2 | |
| | | 3.1 | 30 | 64 | 90 | 1.6 | 7 | |
| | | 4.7 | | | 80 | 1.2 | 8 | |
| | | 4.7 | 30 | 88 | 99 | 3.4 | 12 | |
| A2780/DDP | 2 | | 45 | | 41 | 0.8 | 13 | |
| (1.1) | | 4.7 | | | 15 | 0 | 11 | |
| | | 4.7 | 45 | 50 | 67 | 2.5 | 21 | |
| HOC18 | 27 | | 30 | | 37 | 0.4 | 3 | |
| (6.1) | | 3.1 | | | 41 | 1.0 | 5 | |
| | | 3.1 | 30 | 77 | 81 | >>1.7 | 8 | 2/13 |
| | | 4.7 | | | 81 | >>1.7 | 3 | 1/10 |
| | | 4.7 | 30 | 87 | 87 | >>1.7 | 11 | 3/10 |
| 1A9PTX22 | 34 | | 30 | | 35 | 0.3 | 10 | |
| (7.4) | | 3.1 | | | 38 | 0.3 | 1 | |
| | | 3.1 | 30 | 60 | 67 | 0.8 | 14 | |
| | | 4.7 | | | 57 | 0.6 | 1 | |
| | | 4.7 | 30 | 72 | 75 | >>0.8 | 21 | |

^{a}Tumor Doubling Time in days.
^{b}Cisplatin iv every 7^{th} day for 3 times: ST1926, by oral route, the same day and day after the cisplatin (qdx2/wx3w). *Only two cycles of treatment.
^{c}Tumor volume of treated mice over that of the control mice. In parenthesis the day on which it was evaluated.
Exp: expected as additive effect; Obs: observed experimentally.
^{d}Gross log10 cell kill to reach an established tumor volume (1 cm³). In parenthesis the volume at which it was calculated.
^{e}Body weight loss % induced by drug treatment; the highest change is reported.
^{f}NED, not evidence of disease at the end of the experiment.

TABLE 2

| Dose | | | s.c. growing tumor | | | | i.p. growing tumor | | |
|---|---|---|---|---|---|---|---|---|---|
| (mg/kg) | Route | Schedule | TVI %^{a} | LCK^{b} | BWL^{c} | Tox^{d} | ILS^{e} | LTS^{f} | Tox^{d} |
| 5 × 2 | oral | qdx5/wx4w | 35 | 0.3 | 12 | 0/4 | | | |
| 15 | oral | qdx2/wx2w | 39 | 0.1 | 6 | 0/4 | | | |
| 15 | oral | qdx5/wx3w | 52 | 0.4 | 22 | 0/4 | | | |
| 20 | oral | qdx5/wx2w | n.d. | n.d. | 25 | 2/4 | | | |
| 10 | i.p. | qdx5/wx4w | | | | | 91 | 2/7 | 0/7 |

^{a}Tumor volume inhibition % in treated over control tumors.
^{b}Gross log₁₀ cell kill to reach an established tumor volume.
^{c}Body weight loss % induced by drug treatment, the highest change is reported.
^{d}No. of dead mice/total no. of mice.
^{e}Increase in life span % in treated over control mice.
^{f}Long term survivors at the end of experiments (day 88).

The results indicate a limited activity of ST1926 as single-agent therapy, and in contrast, the combination of the present invention shows a substantial improvement of the anti-tumor effect. An efficacy which is more than additive was achieved in both cisplatin sensitive and resistant tumors, as clearly documented in A2780 and A2780/DDP models. Moreover, an increased efficacy was observed even in the treatment of very responsive tumors (A2780 and HOC-18) with suboptimal doses of cisplatin (3.1 mg/kg). Whereas the treatment with each agent alone produced a marginal activity (<50% TWI), the use of the composition resulted in a significant increase of antitumor activity (TWI in the range of 75-90%). The improvement of efficacy was not associated with an appreciable increase of toxicity (in contrast to the use of optimal doses of cisplatin which caused a body weight loss around 20%).

The observations on the drug ability to inhibit the formation of tumor spheroids in the locoregional treatment (intra peritoneal) of the ascitic IGROV-1 in vitro show that the composition, object of the present invention, is able to control the peritoneal effusion of ovarian carcinoma, which is a common modality of tumor growth in the natural history of the disease in patients.

In conclusion the shown results clearly indicate that, although ST 1926 itself produced a modest antitumor activity against ovarian carcinoma models; its combination with platinum compounds provides a more effective, well tolerated treatment for ovarian carcinoma.

### EXAMPLE 2

Breast Cancer

The MDA-MB231 human breast carcinoma cells (5×10⁶ tumor cells) were injected sc in the right flank of CD1 nude female mice. ST1926 was delivered by oral route (30 mg/10 ml/kg according to the schedule qd×2/w×3w), cisplatin was given intravenously (3.1 mg/10 ml/kg according to the schedule q7d×3). The combination group was treated with both cisplatin and ST1926 (cisplatin was given 1 h before ST1926). Both the chemotherapeutic agents administered alone showed a significative antitumor effect (TVI of 44-50%). When delivered simultaneously, the combination group revealed a significant increase in tumor volume inhibition (TVI was 80%) and in drug persistence on tumor growth (LCK reached a value of 1.34) (FIG. 6).

### EXAMPLE 3

Pharynx Carcinoma

The FaDu human squamous pharynx carcinoma cells (3×10⁶ tumor cells) were injected sc in the right flank of CD1 nude female mice. ST1926 was delivered by oral route (25 mg/10 ml/kg according to the schedule qd×2/w×3w), carboplatin was given intraperitoneally (40 mg/10 ml/kg, according to the schedule q7d×3).

The combination group was treated with both carboplatin and ST1926 (carboplatin was given 1 h before ST1926). Both the chemotherapeutic agents administered alone showed a significative antitumor activity against the xenograft model (TVI was 35% for ST1926 and 52% for carboplatin). When delivered simultaneously, the combination group revealed a significant increase in tumor volume inhibition (TVI was 75%) and in drug persistence on tumor growth (LCK reached a value of 1.2) (FIG. 7).

### EXAMPLE 4

Bone Metastasis

The experimental bone metastasis model of MDA-MB231 intratibially injected (Rucci et al., 2006, J. Pharmacol. Exp. Ther. 318:161-172) is a model used to evaluate the antimetastatic activity of the atypical retinoid ST1926 given by oral route alone and in combination with cisplatin.

Methods

A human breast cancer cell line, MDA-MB231 was cultured in DMEM supplemented with 10% FCS and 1% penicillin-streptomycin solution in a humidified atmosphere of 5% CO2 in air. Cultured used for intratibial injection were at subconfluency. Cells (1×10⁵) were suspended in 0.1 ml of PBS and injected into the tibia of female BALB/c-nu/nu mice with the use of a 27-gauge needle under the anesthesia ip of ketamine and xylazine chloride.

Animals inoculated with tumor cells (day 0) were subdivided in four experimental groups of 7-9 mice/each before starting the treatment 3 days after tumor injection: a group received the vehicle (10 ml/kg of cremophor:ethanol 1:1, further diluted in PBS 1:10), a group ST1926 (25 mg/10 ml/kg, po) according to the schedule qd×2/w×3w, a group cisplatin (3.1 mg/10 ml/kg, iv) according to the schedule q7d×3, a group the combination of the two drugs, where cisplatin was given 1 h before ST1926. Mice were housed inside cages of makrolon (33.2×15×13 cm) with stainless steel cover-feed and sterilized and dust-free bedding cobs. Animals were housed under a light-dark cycle, keeping temperature and humidity constant. Parameters of the animal rooms were assessed as follows: 22±2° C. temperature, 55±10% relative humidity, about 15-20 filtered air changes/hour and 12 hour circadian cycle of artificial light (7 a.m., 7 p.m.). At request, the environmental conditions were monitored and the data are retained in Animal Housing Archives. Drinking water was supplied ad libitum. Each mouse was offered daily a complete pellet diet (GLP 4RF21, Mucedola) throughout the study. The analytical certificates of animal food and water are retained at Sigma-Tau premises. All animals were weighed before starting the experiment and were subdivided into the different dosage groups. Each cage was identified by a paper tag indicating: cage number, group, date of tumor injection, starting date of treatment, name of the test item, dose and route of administration, date of sacrifice.

At the end of the experiment, nude mice were examined by radiography (X-ray) for osteolytic bone metastases, upon mice were subjected to deep anesthesia and X-ray analysis (36 kilovoltage per amperage for 10 s) using a Cabinet-X-ray system (Faxitron model 43855A; Faxitron X-ray Cor. Buffalo Grove, Ill.). Incidence of osteolytic lesions and ostelytic area were evaluated 29 days after tumor implantation.

Results

The treatment with ST1926 was well-tolerated by tumor-bearing mice. Radiographs taken 29 days from injection revealed a high frequency of osteolysis in tibias of vehicle-treated group (78%), whereas no significant differences compared with the control group were revealed in ST1926 (25 mg/kg, po qd×2/w×3w) and cisplatin (3.1, iv q7d×3) treated groups (Table 3, FIG. 8).

By contrast, a significative reduction of osteolytic area (P<0.05) was found in the combination group treated with cisplatin and ST 1926 and also the incidence of osteolytic lesions was lower (22% vs 78%) (Table 3).

These data suggest that a synergistic interaction was observed between ST1926 given by oral route and cisplatin delivered intravenously to prevent the appearance of bone metastases in a human breast carcinoma.

TABLE 3
Presence and burden of lytic lesions in mice with
bone metastastis from MDA-MB231 tumor cells.

| | Incidence of | Osteolytic area | |
|---|---|---|---|
| Group | ostelytic lesions | square pixel ± SD | mm² ± SD |
| Vehicle | 7/9 (78%) | 510 ± 290 | 5.1 ± 2.9 |
| ST1926 | 5/8 (63%) | 310 ± 106 | 3.1 ± 1.0 |
| Cisplatin | 5/7 (71%) | 458 ± 190 | 4.6 ± 1.9 |
| Cisplatin + | 2/9 (22%) | 118 ± 45 | *1.1 ± 0.4 |
| ST1926 | | | |

Treatment started 3 days after intratibial tumor injection. Values are expressed as mean ± SD of relative densitometric units (pixel squares) upon 29 days after tumor implantation.
*P < 0.05 vs the other treated-groups (Mann-Whitney).

On the basis of the teaching of the present invention, and of the results herein presented, the person skilled in the art will be able to prepare a medicament for human use just resorting to the common knowledge of drug development.

1. A method of treating a tumor selected from the group consisting of ovarian tumor, ovarian carcinoma, breast carcinoma and pharynx carcinoma or the treatment of bone metastasis, said method comprising administering an atypical retinoid compound of
formula (I) 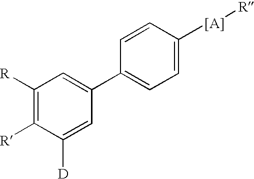
wherein:
R represents alkyl, cycloalkyl, heterocycloalkyl, phenyl, phenyl substituted, adamantyl wherein at least one of the CH can be substituted with C-halogen or C-alkyl and one of the CH₂ can be substituted by O, S, CH-halogen, CH-aryl, CH-heteroaryl, CH-arylalkyl, CH-heteroarylalkyl, CH-amino;
R′ represents OR′″, OCOR′″, COR^{IV};
R-D represents O—(CH₂)ₙ—O; where n=1-3;
D represents H, OH, O-alkyl, (CH₂)ₙ—NH₂, (CH₂)ₙ—NH-alkyl, (CH₂)ₙ—OH, where n=1-4;
R″ represents tetrazole, SO₃H, NHSO₃H, CHO, COOH, CONHOH, CONH-aryl, CONH-C₆H₄OH, CH₂OR′″; PO₃H₂; CO—(CH₂)ₙ-aryl, where n=0-4;
R′″ represents H, alkyl, aryl, arylalkyl, heteroaryl, heteroarylalkyl, SO₃H, α or β D- and L-glycosyl;
R^{IV} represents H, OH, OR′″;
[A] represents [C(R^{V},R^{VI})—C(R^{VII},R^{VIII})]ₙ, [C(R^{IX})═C(R^{X})]ₙ, [C≡C]ₙ, where n=0-3;
R^{V}, R^{VI}, R^{VII}, R^{VIII} represent H, alkyl, halogen, OH, OR′″, NO₂, NH₂, aryl, —O—, —CH₂—, CX₂—, (where X is halogen), —CH(R′″)—;
R^{IX}, R^{X} represent H, OH, halogen, alkyl, aryl, CN, NO₂, COOR′″, their pharmaceutically acceptable salts and solvates;
in combination with an anticancer drug of the platinum family.

2. Method according to claim 1, wherein said compound of formula (I) is selected from the group consisting of:
4-(3-(1-adamantyl)-4-tert-butyldimethyl-silyloxyphenyl)benzaldehyde;
methyl E-4(3-(1-adamantyl)-4-tert-butyldimethyl-silyloxyphenyl)cinnamate;
methyl E-4-(3-(1-adamantyl)-4hydroxyphenyl)cinnamate;
E-4-(3-(1-adamantyl)-4-hydroxyphenyl)cinnamic acid;
methyl 4-(3-(1-adamantyl)-4-methoxyphenyl)propionate;
4-(3-(1-adamantyl)-4-methoxyphenyl)propionic acid;
E-4-(3-(1-adamantyl)-4-methoxyphenyl)cinnamyl alcohol;
methyl E-4-(4-hydroxyphenyl)cinnamate;
methyl E-4-(3-(1-methylcyclohexyl)-4-hydroxyphenyl)cinnamate;
2-(1-adamantyl)-4-bromo-6-N-phthalimidomethyl)phenol;
methyl E-4-(3-(1-adamantyl)-5-(N-phthalimidomethyl)-4-hydroxyphenyl)cinnamate;
E-4-(3-(1-adamantyl)-5-(N-phthalimidomethyl)-4-hydroxyphenyl)cinnamic acid;
E-4-(3-(1-adamantyl)-5-(amino methyl)-4-hydroxyphenyl)cinnamic acid;
4-(7-adamantan-1-yl-benzo(1,3)dioxol-5-yl)-benzaldehyde;
methyl E-4-(7-adamantan-1-yl-benzo(1,3)dioxol-5-yl)-cinnamate;
E-4-(7-adamantan-1-yl-benzo(1,3)dioxol-5-yl)-cinnamic acid;
methyl 2-[4-(3-(1-adamantyl)-4-hydroxyphenyl)]cyclopropanecarboxylate;
cis and trans 2-(4-(3-(1-adamantyl)-4-hydroxyphenyl)]cyclopropanecar-boxylic acids;
methyl E-4-(3-(1-adamantyl)-4-methoxyphenyl)cinnamate;
E-4-(3-(1-adamantyl)-4-methoxyphenyl)cinnamic acid;
and their pharmaceutically acceptable salts and solvates.

3. Method according to claim 2, wherein said compound is E-4-(3-(1-adamantyl)-4-hydroxyphenyl)cinnamic acid.

4. Method according to claim 1, wherein said anticancer drug of the platinum family is selected from the group consisting of cisplatin, carboplatin and oxaliplatin.

5. Method according to claim 3, wherein said combination is E-4-(3-(1-adamantyl)-4-hydroxyphenyl)cinnamic acid and cisplatin.

6. Method according to claim 3, wherein said combination is E-4-(3-(1-adamantyl)-4-hydroxyphenyl)cinnamic acid and carboplatin.

7. Method according to claim 1, wherein said ovarian tumor and/or carcinoma is drug-resistant.

8. Method according to claim 1, wherein said bone metastasis derives from breast cancer.

9. Method according to claim 8, wherein said breast cancer is MDA-MB231.

10. Method according to claim 1, wherein said tumor is selected from the group consisting of HOC 18 ovarian carcinoma, IGROV-I ovarian carcinoma, A2780/DDP ovarian carcinoma, 1A9PTX22 ovarian carcinoma, MDA-MB231 breast cancer, and FaDu squamous pharynx carcinoma.

11. Method of claim 1, wherein the atypical retinoid compound is administered to a human in need thereof in a dosage ranging from about 100 mg/daily to about 1500 mg/daily.

12. Method of claim 1, wherein the atypical retinoid compound is administered to a human in need thereof in a dosage ranging from about 200 mg/daily to about 1000 mg/daily.
